(19) United States
(12) Patent Application Publication
(10) Pub. No.: US 2017/0218446 A1
(43) Pub. Date: Aug. 3, 2017

(54) CELL CHARACTERISATION

(71) Applicant: SISTEMIC SCOTLAND LTD, GLASGOW (GB)

(72) Inventors: Vincent O'BRIEN, Ayr (GB); Chris HILLIER, Glasgow (GB)

(21) Appl. No.: 15/243,202

(22) Filed: Aug. 22, 2016

Related U.S. Application Data
(63) Continuation of application No. 13/818,750, filed on Apr. 11, 2013, now abandoned.

(30) Foreign Application Priority Data
Aug. 23, 2010 (GB) 1014049.9

(51) Int. Cl. C12Q 1/68

(52) U.S. Cl. CPC C12Q 1/6881; C12Q 2600/158; C12Q 2600/178

(57) ABSTRACT

The present invention concerns the finding that non-coding RNA profiles can be exploited as a means of monitoring, assessing, comparing, establishing and/or determining certain cell characteristics and/or profiles. Accordingly, the invention provides the use of non-coding RNA molecules for characterising and/or profiling cells.

### CROSS REFERENCE TO RELATED APPLICATIONS

This is a continuation of U.S. patent application Ser. No. 13/818,750, filed on Apr. 11, 2013, which is a national stage application of International Patent Application No. PCT/GB2011/01241, filed internationally on Aug. 19, 2011, which claims priority to United Kingdom Application GB1014049.9, filed on Aug. 23, 2010, all of which are hereby incorporated by reference in their entireties.

### FIELD OF THE INVENTION

The present invention provides uses of non-coding RNA in methods for characterising and/or profiling cells. In particular, the uses and methods described herein may be exploited to assess the quality, identity, purity, potency and safety of cells and/or cell cultures.

### BACKGROUND OF THE INVENTION

There has been rapid progress in biotechnology and medicine that has led to the development of new treatments and medicinal products, among them products containing viable cells. These new cell-based products have great potential in the treatment of various diseases where there is an unmet medical need. The cell products are, in the case of stem cells, used directly for therapeutic purposes or are research tools to aid drug discovery by providing a homogenous source of stem cells, cells committed to differentiate to one or more lineages or terminally-differentiated cells of a particular lineage. Mammalian cell lines used in research are vital tools for understanding basic biological concepts while cells used in bioprocessing applications can yield macromolecules used for research purposes or clinical applications.

### Current Characterisation and Safety Testing Methods.

There are a number of methods used to assess the quality, consistency and potency of stem cells and cell cultures. For stem cells this is defined as their self-renewal capacity and by the expression of specific markers. The identity of the desired cell population must be defined. Currently hESC lines are characterised using a set of standardised metrics: surface antigens, expression of particular enzymic activities (e.g. Alkaline phosphatase), gene expression, epigenetic markers, assessing genomic stability, cytology and morphology as well in vitro (embryonic body formation) and in vivo differentiation potential (formation of teratoma-like xenografts) and by the absence of measurable microbiological infections. However, the procedures used to assess these stem cell characteristics require skilled staff, but have a relatively low-information-content and are time-consuming and expensive. In addition they do not reveal crucial information on the safety profile and/or fitness-for-purpose of the resultant cells. There is a need for low-skill, low-cost, information-rich QC assays and kits that inform on the quality and consistency of the stem cell lines at derivation and under continued passage in culture, including, for stem cells, expansion of cell populations under conditions supporting proliferation of undifferentiated cells. These QC checks should also provide relevant biological information on their likely suitability for purpose and, if developed for clinical use, their safety for deployment.

There is a requirement to continuously assess the inherent heterogeneity of human-based cell products in order to seek to minimise this variation during the manufacturing of cell-based starting material. Correspondingly, there is a need for a relatively straightforward assay that reports on both phenotypic drift of cells in culture and provides an assessment of the likelihood of their safety profile (e.g. tumourigenicity) if the cells are used as medicinal products.

MicroRNAs (miRNAs) are single-stranded RNA molecules having a length of around 18 to 25 nucleotides. miRNAs were first described by Victor Ambros in 1993 and since then over 2,000 papers on have been published on the subject of miRNAs. There are predicted to be about 1,000 miRNAs in humans, although some estimates place the figure at tens of thousands. miRNA is not translated into protein but instead regulates the expression of one or more genes. Known biology currently shows that microRNAs target particular individual messenger RNAs (mRNAs) or groups of mRNAs, thereby preventing their translation or accelerating mRNA degradation. The mature single stranded miRNA molecule complexes with the RNA-Induced Silencing Complex (RISC) protein and binds to a partially complementary sequence within the 3′untranslated region (3′-UTR) of the protein coding mRNA from its target gene.

Further proteins are recruited to form a silencing complex and the expression of the target gene product is repressed by a mechanism that blocks the translation of the mRNA.

Although much remains to be discovered about the biology of miRNAs and the composition and mechanism of action of the silencing complex it is apparent that miRNAs are involved in the regulation of many genes. MiRNAs are thought to regulate as many as 30% of all genes (Xie et al, 2005) at the translational level. An miRNA can regulate multiple genes and each gene can be regulated by multiple miRNAs permitting complex interrelationships between miRNA/mRNA networks within tissues and cells.

Tissue-specific expression of miRNAs is thought to guide commitment of cells to differentiate and/or actively maintain cell or tissue identity. This wide-ranging influence and interplay between different miRNAs suggests that deregulated expression of a single miRNA or small sub-set of miRNAs may result in striking physiological or pathophysiological changes and complex disease traits (Lim et al, 2005). More than 50% of known human miRNAs reside in genomic regions prone to alteration in cancer cells (Calin et al, 2004). Not surprisingly, the expression pattern of miRNAs change in cancer and other disease states. This information has begun to be used to classify and stage cancers, reveal biomarkers for prognosis and response and provide a critical determinant to guide therapeutic intervention.

An increasing body of evidence confirms that the expression levels of individual miRNAs vary significantly between cell types or within a cell type maintained under different physiological conditions and so can be used to define the cell type, the physiological status of the cell and monitor response to environmental changes.

Embryonic and induced pluripotent stem cells are characterised by their ability to self-renew and differentiate into all cell types. The molecular mechanisms behind this process are complex and rely on the interplay between a network of transcription factors, epigenetic regulators, including miRNAs, and signalling pathways. MicroRNAs play essential roles in maintenance of pluripotency, proliferation and differentiation. Recent studies have begun to clarify the specific role of miRNA in regulatory circuitries that control self-renewal and pluripotency of both embryonic stem cells and induced pluripotent stem cells. These advances point to a critical role for miRNAs in the process of reprogramming somatic cells to pluripotent cells.

We have used the ‘fingerprint’ patterns extracted from the information content held within the miRNA expression profile of cells to monitor the maintenance of cell identity and functional capability. The miRNA profile provides a unique insight into cell biology and can be reduced to practice through the development of kits to monitor pluripotency, cell-fate, cell-identity and phenotypic drift over multiple passages using a single development platform for microRNA screening.

The invention aims to provide alternative methods for monitoring the quality and suitability of cells for the purpose for which they were developed.

### SUMMARY OF THE INVENTION

The invention concerns methods employing non-coding RNA expression assays as a means to characterise cells and/or to monitor the quality and safety profile of in vitro cell culture systems.

Embodiments of the invention include, but are not limited to, determining the non-coding RNA/microRNA profile of cells and serial passages of an in vitro cell culture system. The term “cell” should be understood to encompass any eukaryotic cell. For example a “cell” within the context of this invention may be a mammalian (adult, foetal or embryonic) cell including, for example a stem cell or iPS cell. In one embodiment, a “cell system” according to this invention is (or comprises): (i) pluripotent embryonic stem (ES) cells; (ii) induced pluripotent stem cells (iPS) or ES or iPS cells and/or their intermediate stages differentiating to one or more terminal differentiation states; (iii) adult stem cells (tissue-specific progenitor cells or mesenchymal/stromal cells) or their intermediates differentiating to one or more terminal differentiation states under the influence of external factors in the culture medium; mixtures of cells with varying differentiation profiles; (iv) cell lines used in research or engineered for bioprocessing e.g. for the production of clinical-grade or research grade biological macromolecules. In one embodiment, the “cells” may be fungal cells such as, for example, yeast cells. Cells and cell culture systems may be monitored under optimal growth conditions and/or under conditions where interventions, such as alterations to key element(s) of the growth maintenance regime of the cells is/are altered, so as to determine the affect on the non-coding/microRNA profile of the cell.

The invention reveals sample clustering based on their microRNA expression profile and identifies statistically valid, candidate non-coding/microRNAs which are consistent and reliable markers of undesirable or uncharacterised alterations in the cell system being monitored and therefore provide key decision-support tools on the continued usefulness of the cell system for their intended research, therapeutic or bioprocessing application.

The present invention concerns the finding that non-coding RNA profiles can be exploited as a means of monitoring, assessing, comparing, establishing and/or determining certain cell characteristics and/or profiles. In one embodiment, the various uses and/or methods described herein may be exploited to determine, monitor, establish, compare and/or assess cell characteristics which are also markers of cell quality and/or safety.

Accordingly, and in a first aspect, the present invention provides the use of non-coding RNA molecules for characterising and/or profiling cells.

The inventors have determined that profiles of non-coding RNA molecule expression (referred to hereinafter as non-coding RNA expression profiles) provide a “fingerprint” which can be correlated to, linked or matched with, the presence of particular cell characteristics and/or certain cell profiles. By establishing a non-coding RNA expression profile indicative of one or more cell characteristic(s) or a particular cell profile, it is possible to assess other cells for corresponding characteristics and/or profiles by simple comparison of the non-coding RNA expression profiles. Additionally, the inventors have surprisingly discovered that cells which are shown to be phenotypically identical by standard analytical techniques (such as, for example by flow cytometry and/or cell surface/cytoplasmic/nuclear marker analysis and the like) can be shown by the micro-RNA profiling techniques described herein, to be genotypically (and thus most likely phenotypically) distinct/different. Where cell safety and quality are concerned, the phenotypic differences between an un-safe (for example tumorogenic) cell or cells and/or a cell of poor quality (perhaps lacking expression of specific markers), may be undetectable by standard techniques.

The instant invention provides a highly sensitive an accurate means of establishing whether or not a cell or cell system (for example the population of cells within a cell culture) conforms to a set of predetermined standards. One of skill will appreciate that provided one establishes a micro-RNA profile of a cell which is known to conform to a set of predetermined safety and/or quality standards, other cells of the same type can be assessed for conformity with the predetermined safety and/or quality standards by comparison of micro-RNA.

In view of the above, one embodiment of this invention, provides the use of non-coding RNA molecules for characterising and/or profiling cells, wherein the cells are shown to be phenotypically identical to a reference cell by methods other than micro-RNA profiling. In one embodiment, the method by which the cell and a reference cell are shown to be identical may be flow cytometry. In this context, a reference cell may be a cell conforming to a predetermined set of safety and/or quality standards.

In one embodiment, the methods provided by this invention may exclude methods which exploit micro-RNA profiling to distinguish one differentiative cell state from another. For example, in some embodiments, the invention may not embrace the use of micro-RNA profiling to assess the differentiation of stem cells to other cell types.

A second aspect of this invention provides a method of characterising and/or profiling a cell, said method comprising the steps of comparing the non-coding RNA profile of said cell with a reference non-coding RNA expression profile. In one embodiment, the reference non-coding RNA expression profile may be derived from a cell possessing characteristics and/or a profile which should be present and/or exhibited by the cell being characterised/profiled.

It should be understood that a cell “characteristic” or “profile” may relate to cell features such as identity (type), morphology, genotype, phenotype, viability, potency (for example degree of pluripotency), contaminant levels, safety (for example tumourigenicity) and/or quality. In certain embodiments, a cell “profile” may be determined by establishing aspects of one or more of a cell's morphology, genotype, phenotype, viability, potency (pluripotency), contaminant levels, safety (tumourigencity) and/or quality. One of skill will appreciate that the terms cell “characteristic” and/or “profile” may relate to the biological activity and/or compound secretion/production profile. By way of example, a cell characteristic and/or profile may relate to the ability of a cell to express, produce and/or secrete a natural of heterologous compound or compounds such as, for example, a protein, peptide, amino acid, nucleic acid, carbohydrate and/or other small organic compound.

The term “non-coding RNA” may include microRNA (miRNA) molecules and either or both miRNA precursors and mature miRNAs. The term may further include small interfering RNAs (siRNA), piwi-interacting RNAs (pi RNA), small nuclear RNA (snRNA) and short hairpin RNA (shRNA). “Non-coding RNA” according to this invention may further comprise transgenic non-coding RNAs which may function as reporters of non-coding RNA expression. The non-coding RNAs may be episomal and the methods and/or uses described herein may require initial steps in which episomal DNA is introduced into the cells described herein whereupon the episomal DNA can be transcribed to produce non-coding RNA which constitutes all or part of the profiled non-coding RNA. In one embodiment, the term “non-coding RNA” does not include non-coding RNAs known as “teloRNA” or “teloRNA mark”.

A non-coding RNA expression profile may relate to the expression and/or identity of at least one non-coding RNA. In one embodiment, the non-coding RNA expression profile relates to the expression of a plurality of non-coding RNAs. Accordingly, a non-coding RNA expression profile may comprise some indication of the identity of one or more non-coding RNAs expressed by a cell optionally together with quantitative and/or qualitative measurements of the level of expression of one or more non-coding RNAs within a cell.

In certain embodiments, the methods and uses described herein may require the use of a non-coding RNA expression profile database. Such a database may be referred to as a non-coding RNA reference library. Non-coding RNA databases described herein may comprise one or more reference non-coding RNA profiles each being derived from a cell having known characteristics/profiles and/or cells which have been cultured according to a particular protocol and/or subjected to known or defined interventions.

In one embodiment, the reference non-coding RNA profiles may be derived from an isolated cell, cells derived from a cell culture, cell line and/or stored cell preparation. Additionally or alternatively, the reference non-coding RNA profiles may be obtained from cells subjected to one or more defined or predetermined interventions and/or cells subjected to a particular culture protocol, altered culture conditions and/or one or more interventions. The reference non-coding RNA profiles described herein, may comprise non-coding RNA profiles derived from single cell types and/or a plurality of different cell types. In other embodiments, the reference non-coding RNA profile may be derived from primary cell cultures and/or immortalised cells. Advantageously, the reference non-coding RNA profile is obtained from a cell or cell exhibiting known and/or desired characteristic(s), a desired and/or correct profile and/or an a cell or cells which meet a certain predetermined quality and/or standard.

Since the reference non-coding RNA expression profiles are derived from cells exhibiting known (desirable) characteristics and/or profiles, one of skill will appreciate that any cell which exhibits a comparable non-coding RNA profile, must possess similar characteristic(s) or a similar profile.

The reference non-coding RNA expression profiles may be compiled using multiple sets of data obtained from repeat non-coding RNA expression analysis of cells having known characteristics and/or known profiles and/or from non-coding RNA expression analysis of cells conforming to known or approved standards.

For convenience, the reference micro-RNA profiles described herein may be referred to as “comparative micro-RNA profiles”.

The process of comparing non-coding RNA expression profiles obtained from cells to be characterised, profiled and/or quality assessed, with reference non-coding RNA profiles (optionally contained within a database) as described herein, may involve identifying correlations between non-coding RNA profiles. Correlations between non-coding RNA profiles of cells being characterised, profiled and/or quality assessed are typically correlations, positive or negative, between changes in the expression of one or more non-coding RNAs. For example, a positive correlation may comprise the identification of a particular non-coding RNA profile in a cell being characterised, profiled or quality controlled and the same non-coding RNA profile in reference non-coding RNA profile (or database). A negative correlation may comprise the identification of a particular non-coding RNA profile in a cell being characterised, profiled or quality controlled and a reference non-coding RNA profile which, while exhibiting expression of corresponding non-coding RNAs—exhibits variable or differential expression levels (i.e. the expression of a particular non-coding RNA in a reference profile may be less than when compared to the expression of the same non-coding RNA identified in a cell being characterised, profiled and/or quality controlled).

The reference non-coding profiles and/or databases described herein may comprise non-coding RNA expression profiles which have been categorised (clustered or grouped) on the basis of similarities present in the reference non-coding RNA profiles. For example, data relating to particular cell types and/or to cells cultured in a particular way, may be grouped together so as to facilitate probing a database for correlations with non-coding RNA profiles of cells being characterised, profiled and/or quality controlled.

In view of the above, the non-coding RNA profiles contained within the reference non-coding profiles provided by this invention may represent the profiles of one or more types of cell, cells at various stages of culture, cells cultured according to particular protocols and/or cells subject to one or more interventions—perhaps an intervention occurring during culture.

The term “intervention” may be taken to include the act of administering a compound or compounds to a cell. In other embodiments, an intervention may include the change of culture media, the addition of one or more media supplements as well as alterations in culture conditions such as, for example, time, temperature, pH and/or osmolality. An intervention may also include the transfer of cells from one culture vessel to another—perhaps as a result of cell sub-culturing procedures.

The present invention finds particular application in the field of cell culture where it may be necessary to ensure that one or more cell interventions or protocols has not had a deleterious effect on the cells of the cell culture. For example, by compiling a reference non-coding profile of cells which exhibit favourable or desired characteristics before during and/or after successful culture according to one or more protocols, it may be possible to establish whether other cells cultured according to the same protocols exhibit the same characteristics before, during and/or after culture, by simple comparison of non-coding RNA profiles.

Where the reference non-coding RNA profiles are intended to represent the characteristics and/or features of cells being cultured, non-coding RNA profiles may be obtained from serially passaged (split and/or subcultured) cultures of cells either at or during each passage and/or at various other points during culture. Additionally, or alternatively, when culture conditions are altered or the cells of the culture are subject to an intervention (perhaps the addition of a supplement (antibiotic, nutrient or the like), a reference non-coding RNA expression profile may be obtained.

In this way, it is possible to construct a database comprising one or more reference non-coding RNA profiles which reflect the non-coding RNA profiles of cells in culture. One of skill will appreciate that such a database may be used to monitor and/or assess cell cultures by comparison of the non-coding RNA profiles of cells from the cell culture with the reference non-coding RNA profiles of the database.

In one embodiment, the methods provided by this invention may be used to assess the effect of specific culture substrates (or components thereof) on cells and cell cultures. For example, the methods of this invention may be exploited as a means of assessing or monitoring the performance of nanofibres/nanoscale growth surfaces which can be used to maintain the pluripotency or a specific differentiative state of stem cells. In such cases, a micro-RNA profile indicative of a pluripotent cell or correctly differentiated cell would be obtained and compared to the micro-RNA profile of cell cultured on a nanofibres/nanoscale growth surface in order to determine whether or not the cells remain pluripotent or correctly differentiated.

In other embodiment, the micro-RNA profiling methods provided by this invention may be exploited to assess the effectiveness of a lyophilisation technique or the viability of cells subjected to such a process. Again, comparative micro-RNA profiles would be obtained from cells before and after a lyophilisation process and/or cells which remain viable after lyophilisation. Such techniques could be applied to erythrocyte lyophilisation protocols.

In yet further embodiments, the micro-RNA profiling provided by this invention may be used to assess the effectiveness of protocols which force the differentiation of one cell type from another. Such protocols may include those which cause differentiation without a pluripotent intermediate. By way of example, the micro-RNA profiling methods of this invention may be used to assess the success of a fibroblast/erythrocyte differentiation protocol, a comparative micro-RNA profile being obtained from a correctly differentiated erythrocyte cell.

Non-coding RNA expression profiles may be measured or determined for each non-coding RNA within a particular group or subset of non-coding RNAs. Additionally, or alternatively, non-coding RNA expression profiles may comprise the identification of an individual non-coding RNA and measuring and/or determining the expression thereof.

The level of expression may be determined indirectly via measurements of the amount or level of activation of a reporter construct, for example a transgenic reporter construct incorporated into the genome of a cell.

The methods and uses of this invention may find particular application in cell quality control and/or safety analysis procedures. One of skill in this field will appreciate that commercial production, sale and distribution of cells—particularly cells derived from stored cell lines, is subject to stringent quality and safety control, primarily to ensure that stored cells and/or cells distributed to customers, meet certain predetermined standards. For example it may be necessary to ensure that cells cultured from stored cell lines are as described (both in terms of identity and morphology), are viable and exhibit certain characteristics (features and/or traits).

Current cell quality control processes or procedures, may involve a series of complex, time consuming and costly tests—each of which is designed to confirm that a cell meets a pre-determined standard. Such tests may be performed prior to shipping a cell line to a customer but also at regular intervals during storage or culture. By way of example, cell quality control procedures may comprise tests designed to assess cell identity/morphology, cell phenotype, cell genotype, levels of cell contamination, degree of pluripotency, cell viability and/or cell safety. Such tests may involve the use of DNA profiling techniques, immunohistochemistry, alkaline phosphatase staining, flow cytometry, gene expression analysis (perhaps using expression arrays and the like), blood group typing, karyology, microorganism screening (using PCR and immunological based techniques), teratoma and embryoid body formation (particularly relevant where the pluripotency of a stem cell is being tested) and simple live/dead (trypan blue) stains to determine viability.

By establishing a reference or comparative non-coding RNA profile indicative of a certain cell “standard” or “quality standard”, it is possible to quality control cells by comparison of non-coding RNA profiles. By way of example, the non-coding RNA profile of a cell cultured from a stored cell line may be compared with the non-coding RNA profile (i.e. a reference non-coding RNA profile) of the same type of cell which is known to meet one or more predetermined standards. If the non-coding RNA profile of the cell being cultured is comparable to, or matches with, the (reference) non-coding RNA profile derived from a cell known to meet one or more pre-determined standards, one may conclude that the cultured cell meets the same standards.

It should be understood that the term “standard” or “quality standard” may relate to defined criteria or features which any given cell must exhibit prior to being used (in anyway whatsoever), sold or distributed. Such standards may be set by regulatory bodies but may also relate to locally determined cell features and/or characteristics which render cells suitable for particular uses—for example uses in assays and the like.

In view of the above, the present invention provides use of non-coding RNA profiles in cell quality control.

In a further embodiment, the invention provides a method of quality controlling cells, comprising the steps of comparing the non coding RNA profile of cells to be quality controlled, with a reference non-coding RNA profile. In one embodiment, the reference non-coding RNA profiles may be derived from a cell or cells known to meet a certain quality standard. Since the reference non-coding RNA profiles are derived from a cell meeting one or more predetermined standard(s), any cell which exhibits a non-coding RNA profile corresponding to a reference non-coding RNA profile, must be of a similar quality standard. In one embodiment, the non-RNA profile of the cell to be quality controlled may be compared with a database comprising one or more reference non-coding RNA profiles.

In one embodiment, the quality control procedures comprise establishing the identity, phenotype, genotype, levels of contamination, viability and/or pluripotency in stored and/or cultured cells.

Advantageously, the reference non-coding RNA profiles described herein may be derived from cells of known identify and having defined phenotypes and/or genotypes, known levels of contamination (low/no contamination, moderate or high levels of contamination), defined pluripotency (for example complete, partial or no pluripotency), and defined levels of viability.

For example, methods for assessing the pluripotency of a cell may comprise the step of comparing the non-coding RNA profile of a cell with unknown pluripotency with the non-coding RNA profile of the same type of cell having a known level of pluripotency.

Similarly, cell identity may be confirmed by comparing the non-coding RNA profile of a cell (perhaps a cell of unknown identity) with the non-coding RNA profiles of a cell of known identity. If the non-coding RNA profile of the unknown cell corresponds to, or matches with, the non-coding RNA profile of any of the known cells, then it may be concluded that the unknown cell is the same as the cell from which the corresponding or matching non-coding RNA profile was derived.

In one embodiment, the methods described herein may be exploited to establish a level of Mycoplasma contamination in a cell or cells. One of skill will appreciate that a comparative or reference micro-RNA profile may be obtained from a corresponding cell type or cell population known to be free from Mycoplasma contamination.

One of skill will appreciate that the present invention, and in particular those embodiments relating to cell quality control, finds particular application in the field of cell culture, particularly commercial cell culture where large numbers of cells are stored and cultured.

When culturing cells, it is often important to make regular checks to ensure that the cultures comprise cells which meet certain predetermined standards. For example, beyond establishing that the cultured cells are of the correct cell type, it may be necessary to ensure that the cell expresses certain markers or that the cell expresses a particular compound or compounds or that interventions which occur during cell culture do not have a deleterious effect upon the cells. Where the cell culture comprises stem cells, it may be necessary to ensure that the cells of the culture comprises cells which remain pluripotent throughout passage and/or that the cell follows a particular differentiation path. By comparing the non-coding RNA profiles of cultured cells with the non-coding RNA profiles of cultured cells conforming to known or predetermined culture standards, it is possible to ensure that the cells being cultured meet those same standards.

In one embodiment, a database comprising one or more reference non-coding RNA profiles may comprise non-coding RNA profiles obtained from cells being serially passaged and at various stages of culture. For example, the database may comprise the non-coding RNA profiles of one or more different types of cells during early-, mid- and/or late-phase passage or culture or at any other time point there between. Additionally or alternatively, the database may contain the non-coding RNA profiles of cells which have been subjected to some form of altered culture condition (for example altered time, temperature, pH, nutrient and/or metabolite availability). In other embodiment, the database may contain non-coding RNA profiles obtained from one or more cells which have been contacted with various agents such as, for example, growth media supplements including, vitamins, nutrients, nucleic acids, antibiotics, candidate drug compounds, test agents, antibodies, carbohydrates, proteins, peptides and/or amino acids. It should be understood that the database may contain many such non-coding profiles obtained from a variety of different cell types.

One of skill will appreciate that the data comprising the reference non-coding RNA profiles may be compared with data from cells being tested, with the aid of data processing/analysis techniques such as, for example statistical mathematical methods. For example, techniques such as principle component analysis or pattern recognition algorithms may be used to identify correlations between data contained within the database and non-coding RNA expression profiles obtained from cells being tested.

In other aspects, the invention may provide a kit for characterising, profiling and/or quality controlling cells, said kit comprising a database of one or more reference non-coding RNA profiles and assay systems, apparatus and/or reagents necessary to obtain non-coding RNA profiles from cells to be characterised, profiled and/or quality controlled. The user may simply obtain the non-coding RNA profile of a cell to be characterised, profiled and/or quality controlled and simply compare the non-coding RNA profile with the non-coding RNA profile(s) of the database.

In a further aspect, the present invention may relate to a cell characterisation, profiling and/or quality control service whereby a service provider receives cells from third parties to be characterised, profiled and/or quality controlled. The service provider may have one or more non-coding RNA databases of the type described herein and which can be used to compare the non-coding RNA profiles of the cells provided by the third parties. Once the non-coding RNA profiles of the cells provided by the third parties have been compared with the non-coding RNA profiles of database, the third party may then be provide with a report detailing information relating to the characteristics, profile and/or quality of the cells.

Such a service may be particularly useful to third parties involved in cell storage and/or culture. The service may be of particular use to those who are required to make regular checks of cells in storage or culture to determine cell identity/type, cell phentotype/genotype, viability, pluripotency, levels of contamination and the like. Furthermore, the services described herein may be used to ensure that cells subjected to particular interventions or culture protocols possess the required characteristics before, during and after execution of the protocol and/or intervention.

The third party may further provide information relating to the culture protocols used to culture the cells and/or information relating to certain features, traits and/or characteristics the cells to be characterised, profiled and/or quality controlled, should have.

### DETAILED DESCRIPTION

The present invention will now be described in detail with reference to the following figures which show:

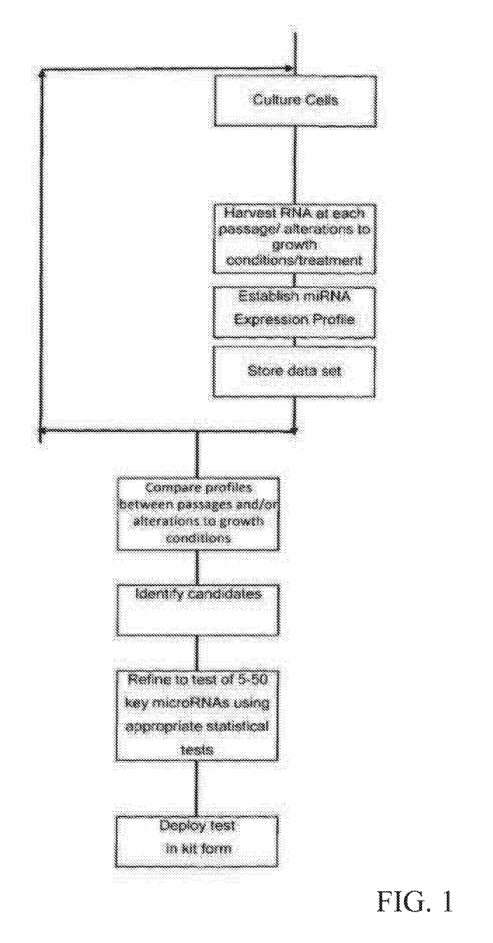

FIG. 1 is a flow diagram of a method according to the invention;

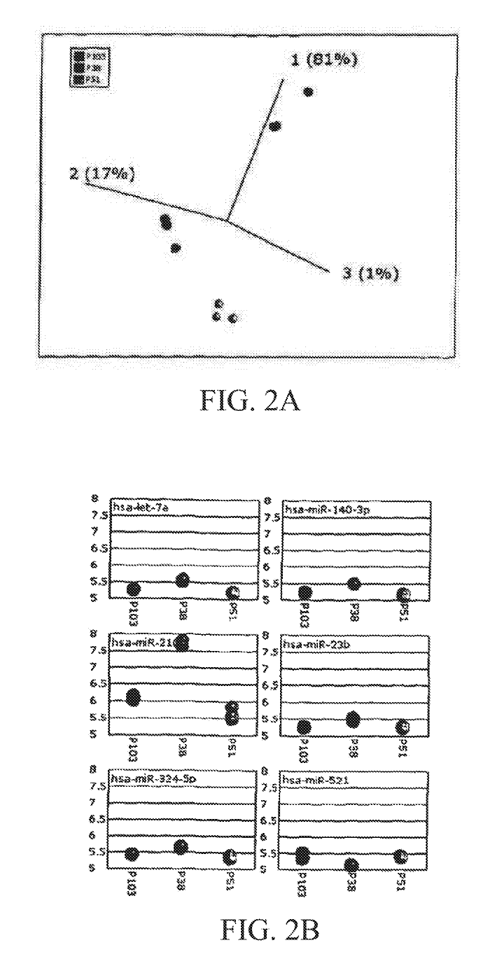

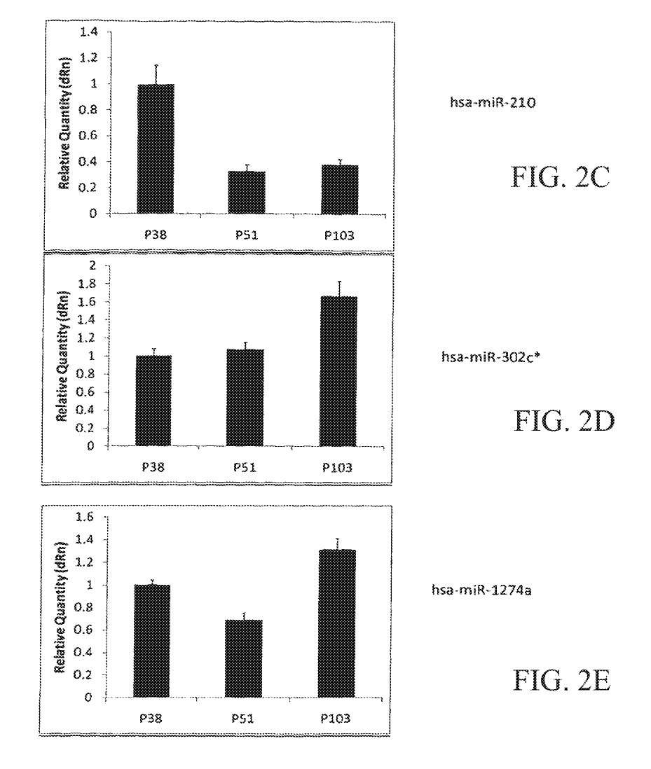

FIGS. 2A-2E are graphs showing decreased expression of hsa-miRNA-210 and increased expression of hsa-miR-1274a and hsa-miR-302c* with extend in vitro passage of hESCs with both microarray and QPCR data panels; FIG. 2A shows a Principal Components Analysis revealing separation of samples based on cell passage number in human embroyonic stem cell line RCM1; FIG. 2B shows an expression profile analysis of micro RNA microarray expression data (normalised signal intensities from the array) for hsa-miR-210 and three other microRNA which do not significantly change expression between passages; and FIGS. 2C-2E show confirmation of key microRNA expression differences by qRT-PCR data;

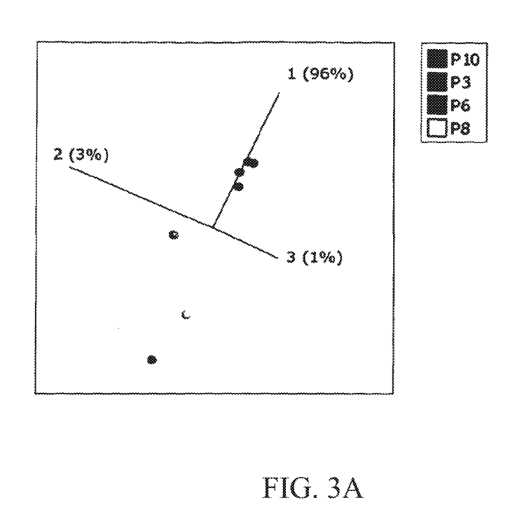

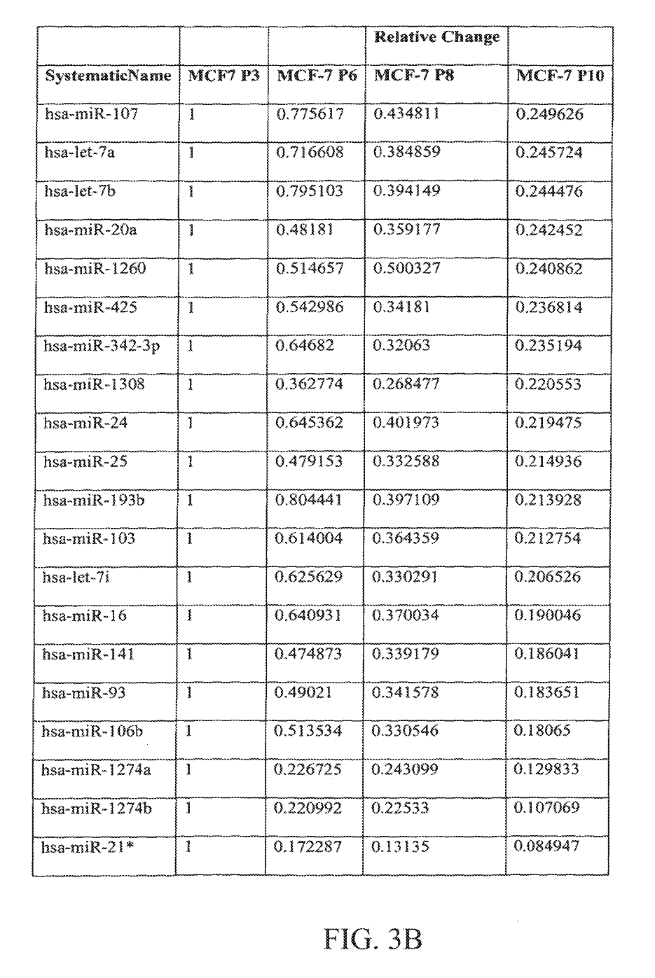

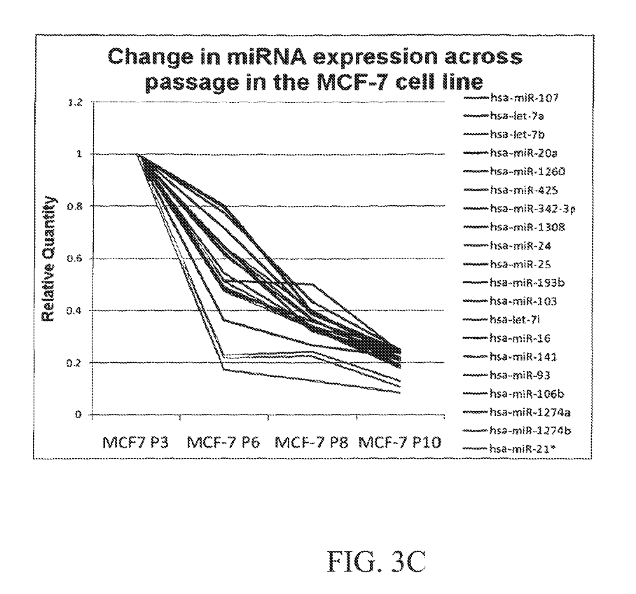

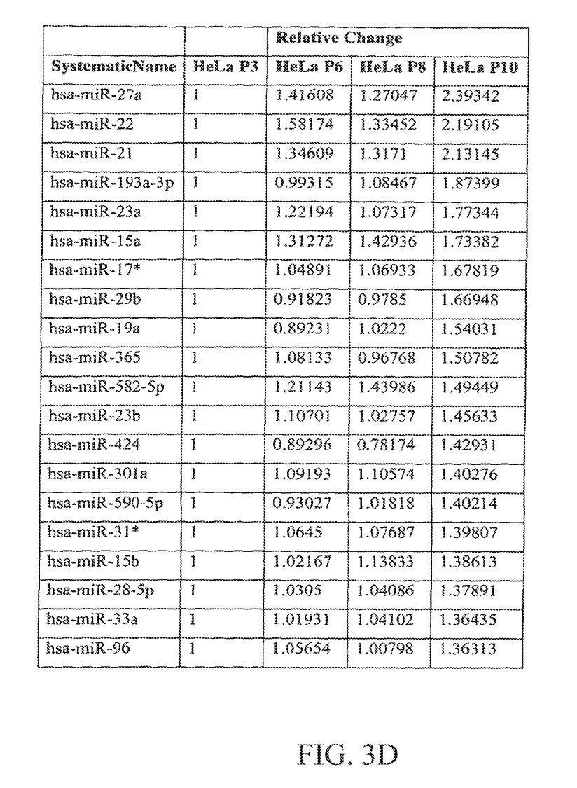

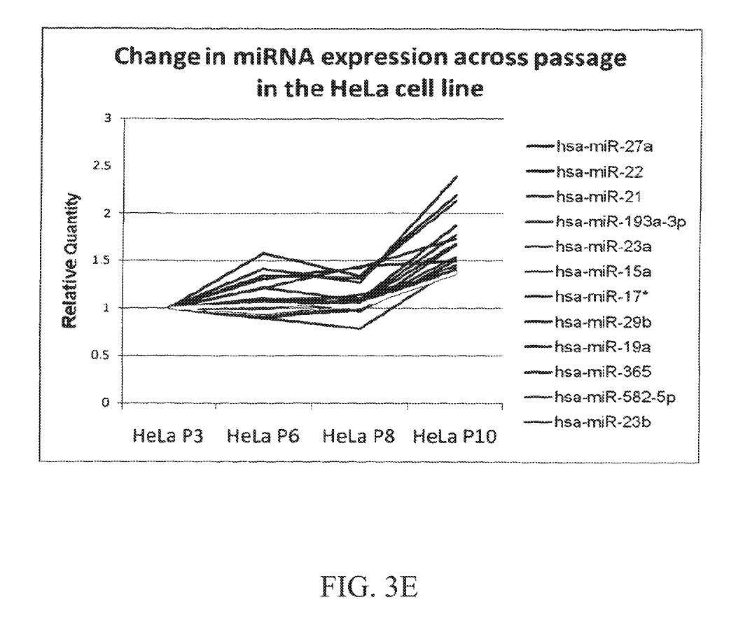

FIGS. 3A-3E show phenotypic ‘drift’ of human cancer-derived cell lines (HeLa and MCF-7) with extended passaging in vitro; FIG. 3A is a graph showing alterations in microRNA profiles in a serially passaged human, tumour-derived cell lines (HeLa and MCF-7); principal components analysis of microRNA datasets reveals separation of samples based on cell passage number in MCF-7 cells; FIG. 3B is a table showing twenty miRNAs altered during serial passage of MCF-7 cells in culture. All twenty miRNAs show significant decreases in gene expression over the seven passages monitored. The changes are shown as relative changes (fold changes) in comparison to the earliest passage (P3) cells. FIG. 3C shows a profile analysis of the twenty miRNAs of FIG. 3B; FIG. 3D shows twenty miRNAs altered during serial passage of HeLa cells in culture. All twenty miRNAs show significant alterations in miRNA expression over the seven passages monitored. The changes are shown as relative changes (fold changes) in comparison to the earliest passage (P3) cells. FIG. 3E shows a profile analysis of the twenty miRNAs of FIG. 3D;

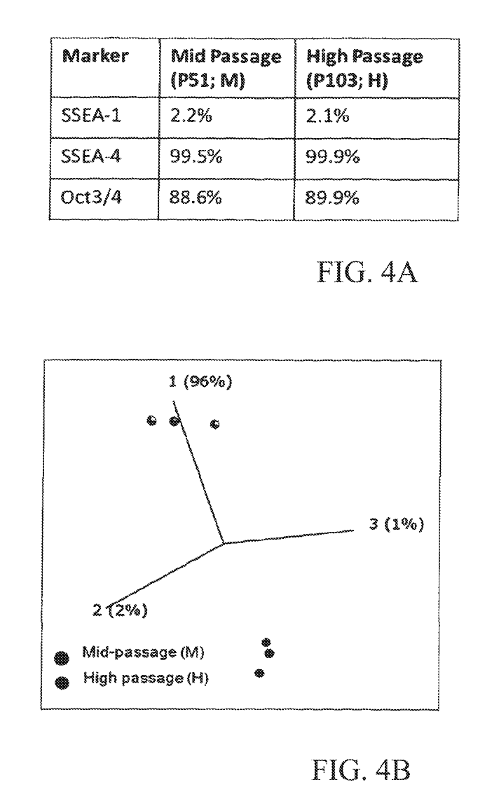

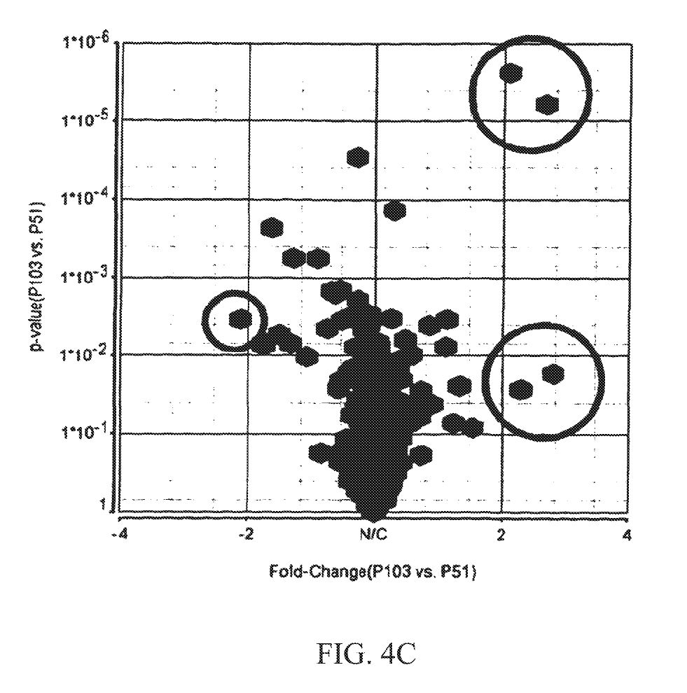

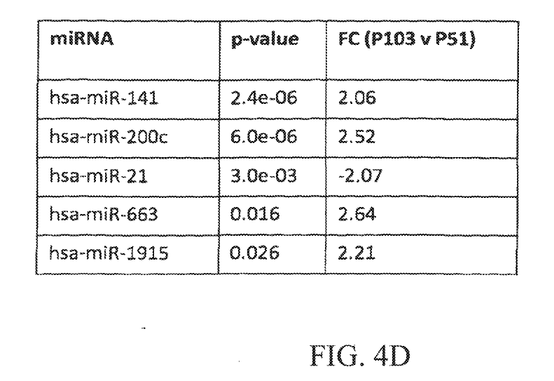

FIG. 4A shows Flow Cytometry results for 2 hESC populations that are maintained under identical culture conditions for extended passages;

FIG. 4B shows principal component analysis (PCA) of miRNA profile of the Mid- and High-passage hESC populations;

FIG. 4C shows a volcano plot representing the differential expression of microRNA between mid-passage (P51) and high-passage (P103) cells. The 5 differentially-expressed miRNAs with a fold-change difference of 2 or more are circled in red;

FIG. 4D shows the identification of 5 microRNAs (circled red in FIG. 4C) which demonstrate a greater than 2-fold differential expression between P51 and P103 hESC cultures; and

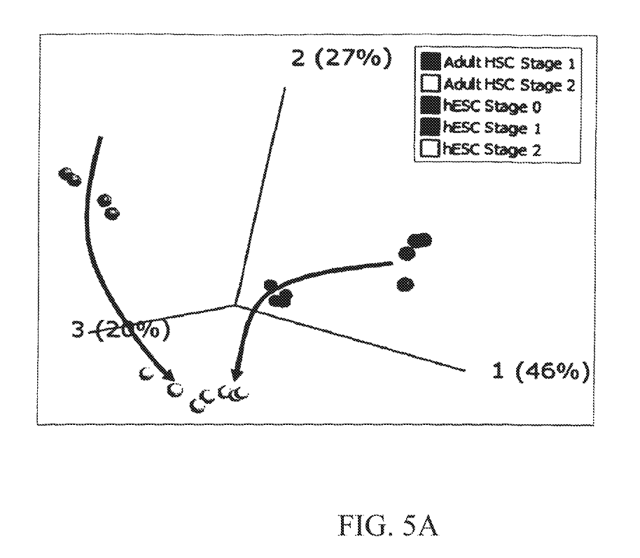

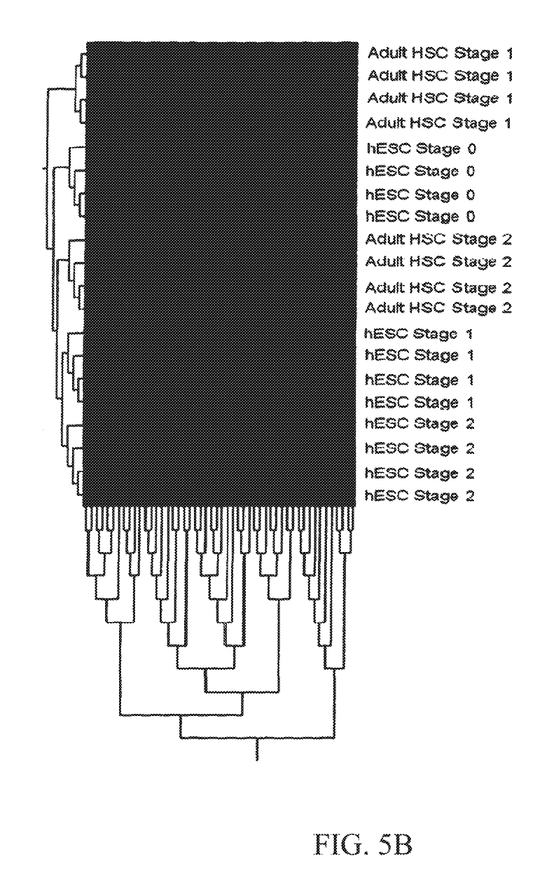

FIGS. 5A and 5B are graphs showing clustering of different sample groups based on differences in miRNA expression profiles. A. FIG. 5A shows a principal component analysis (PCA) where the arrows denote the trajectories of differentiation; B. FIG. 5B show sample relationships using hierarchical clustering and a heatmap.

### EXAMPLE 1

In an example application of the invention, a database of miRNA expression data sets (being an example of an expression data set derived from a measured non-coding RNA expression profile) are prepared. With reference to FIG. 1, suitable human embryonic stem cells are cultured by known methods over an extended period of time and sampled at 3 points after their derivation i.e. at passages 38, 51 and 103. A miRNA expression profile is then measured using a sample of the cells at each passage to determine the expression level of each of a number of miRNAs in the treated cells.

Two alternative methods for measuring the miRNA expression profiles, microarray analysis and qualitative real-time PCR analysis, are set out below.

(1) miRNA Microarray and Data Analysis

Total RNA from reference cells (n=3) is isolated using a column-based kit from Exiqon A/S of Vedbaek, Denmark. Two μg of total RNA from each sample is analysed by miRNA microarray. miRNA microarray analysis including labelling, hybridization, scanning, normalization and data analysis is commercially available from a number of sources, for example, from Exiqon A/S. Briefly, RNA Quality Control is performed using Bioanalyser 2100 microfluidics platform (Bioanalyser is a trade mark of Agilent Technologies). Samples are labelled using the Complete Labelling Hyb Kit from Agilent, following the provided instructions.

(2) Quantitative Real-Time PCR

As with option (1) above, all cellular RNA is extracted using a column-based kit from Exiqon and following the manufacturer's instructions. Quantification of miRNAs by TaqMan Real-Time PCR is carried out as described by the manufacturer (Applied Biosystems of Foster City, Calif., USA). (TaqMan is a trade mark of Roche Molecular Systems, Inc.). Briefly, 10 ng of RNA is used as a template for reverse transcription (RT) using the TaqMan MicroRNA Reverse Transcription Kit and miRNA-specific stem-loop primers (Applied Biosystems). An aliquot (1.5 μl) of the RT product is introduced into 20 μl PCR reactions which are incubated in 96-well plates on the ABI 7900HT thermocycler (Applied Biosystems) at 95° C. for 10 min, followed by 40 cycles of 95° C. for 15 s and 60° C. for 1 min. Target gene expression is normalized between different samples based on the values of U48 RNA (a small, non-coding RNA) expression (or U6 RNA, if U48 is found to vary between samples).

Experimental Findings and their Implications.

Using the methods described we have established that it is possible to determine a novel way to monitor the identify the phenotypic drift of cells based on the grouping of miRNA expression data. Furthermore, the method can be employed to identify certain miRNAs, having expression levels which are indicative of potential alterations in cellular functions including pluripotentcy and tumourigenicity. These miRNAs will enable future intervention screening to analyse a relatively small group of miRNA expression levels changes to identify key alterations in cell physiology/pathophysiology with specific subsets, and not the entire miRNA repertoire, being used depending on the particular endpoint being investigated.

An example of using a select small group of miRNAs to determine potential Safety of a human embryonic stem cell population is given below.

Materials and Methods

RCM1 Cell Culture.

Derivation

The cell line RCM-1 was derived from a freshly received Day 6 Blastocyst. It was manually hatched using a Swemed Stem Cell cutting tool (Vitrolife AB, Cat No: 14601) and the inner cell mass isolated and plated onto human fibroblasts (Cascade Biologies). The fibroblasts had been pre-plated onto tissue culture wells which in turn had been pre-coated with a layer of human Laminin (Sigma, Cat No: L4544). The cells were cultured in conditioned medium containing 24 ng/ml human basic fibroblast growth factor (hbFGF) (Invitrogen, Cat No: PHG0261). The resultant outgrowth was manually passaged using a Swemed Stem Cell cutting tool and through early expansion continued to display a typical undifferentiated morphology while on the laminin/feeders plus hbFGF culture system.

The characteristics of the cell line represented in the summary document available online at http://www.roslincells.com/sitepix/downloads/RCM-1.pdf

Expansion

RCM-1 was then adapted to a feeder-free culture system of CellSTART matrix (CS) (Invitrogen, Cat No: A10142-01) with StemPRO (SP) (Invitrogen, Cat No: A1000701) medium containing 8 ng/ml hbFGF and under these conditions has maintained an undifferentiated morphology. The cell line was expanded through a number of passages using mechanical/manual methods in preference to enzymatic methods. At various passage stages, during the expansion of the cell line, cells were cryopreserved, as described and following manufactures instructions, using CryoStor CS10 (Stemcell Technologies, Cat No: 07930). Recovery from Cryopreservation

Three passage time-points, early, mid and late were thawed for the study, namely passages P38, P51 and PI 03.

Vials, in triplicate, were removed from −150° C. freezer and quickly thawed at 37° C. The thawed cells were them washed twice in pre-warmed medium before being resuspended in fresh pre-warmed medium and plated into wells in a culture system of CellSTART matrix (CS) (Invitrogen) with StemPRO (SP) media containing 8 ng/ml hbFGF.

Cells were cultured for 7 days (FIG. 1), with repeated medium changes, before harvesting for RNA extraction (see below).

Flow Cytometry Analysis

The cells which were harvested for RNA extraction were also sampled to determine the expression of the multiple markers of pluripotency and differentiation.

A single cell suspension was made from the remaining cells in culture and stained for the various markers associated with either a differentiated or undifferentiated state. The markers stained for were: stage-specific embryonic antigen 1 (SSEA-1) where an up regulation is indicative of a differentiated state, stage-specific embryonic antigen 4 (SSEA-4) where an up regulation is indicative of an undifferentiated state and Oct3/4, a 34 kDa POU transcription factor that is expressed in embryonic stem (ES) cells and germ cells, and its expression is required to sustain cell self-renewal and pluripotency, using a Human and Mouse Pluripotent Stem Cell Analysis Kit (BD, Cat No: 560477).

The stained cells are analysed using Flow Cytometry and the results produced give the status of the cell line both numerically and graphically for the markers analysedas shown in FIG. 4A.

Tumour-Derived Cell Lines

HeLa and MCF-7 cells were cultured and passaged (sub-cultured) using standard methods.

RNA Extraction

Prior to miRNA profiling analysis, total RNA must be isolated from the cells, and analysed for quality. Total RNA from stem cells, at different passage numbers, is isolated using the miRCURY RNA isolation kit, obtainable from Exiqon (Denmark). Following the manufacturer's instructions, the cells are lysed in the tissue culture dish using a specific lysis buffer, and transferred to a column where the RNA is washed then eluted. RNA quantity and quality is checked using the Nanodrop ND-1000 spectrophotometer (Thermo Fisher of Waltham, Mass., USA) and the Bioanalyser 2100 microfluids-based platform (Agilent Technologies of Santa Clara, Calif., USA).

Micro RNA expression profiles for stem cell samples of different passage numbers can be determined by isolating total RNA from these samples and analysing them by two methods; (1) miRNA microarray and:

(2) Quantitative Real-Time PCR (QPCR).

Microarrays are used to achieve a complete miRNA profile of a sample, by collecting data on the expression levels of human 851 miRNAs simultaneously. QPCR is used to interrogate an individual miRNA of interest in a number of samples so differences in expression levels can be determined.

(1) miRNA Microarray and Data Analysis

Total RNA that has been checked for quality and has been diluted to an appropriate concentration is used as the starting material for miRNA profiling on the Agilent microarray platform. 100 ng of total RNA from each sample is processed through the microarray protocol, in which the microRNAs are labelled, hybridised to an array and scanned using the Agilent Microarray Scanner. Samples are labelled with Cy3 dye using the Agilent ‘miRNA Complete Labeling and Hyb kit’ and hybridised overnight on an Agilent miRNA array, 8 of which are found on each glass slide. On an array, each miRNA is represented 16 times, by at least 2 different probes. In addition, spike-in controls are used to evaluate the labelling and hybridisation efficiency of the reactions. Scanned images of the arrays constitute the input for the Agilent Feature Extraction software, which analyses each spot on the image, assigning it to a specific miRNA and calculating a value for the emitted fluorescent signal. The output from this processing is a series of QC reports, which evaluate the quality of the array processing, and text files, which contain the raw microarray data. These text files form the basis of the statistical analysis which is used to identify changes in miRNA expression between different samples. For best experimental design, biological replicates (n=3) are processed on different slides to ensure reproducibility. Microarray data is interpreted by statistical analysis programs such as GeneSpring (Agilent Technologies) and/or Omics Explorer (Qlucore of Lund, Sweden), and by Sistemic's in-house statistical methods (see below).

RNA Extraction

RNA was isolated and purified from these cells using a column-based kit from Exiqon the following procedure. The medium the cells were grown on was aspirated and the cell monolayer was washed with an appropriate amount of PBS. The PBS was further aspirated. 350 μL, of the lysis solution was added directly to a culture plate. The cells were lysed by gently tapping the culture dish and swirling buffer around the plate surface for five minutes. The lysate was then transferred to a micro-centrifuge tube. 200 of 95-100% ethanol was added to the lysate and mixed by vortexing for 10 seconds. A column was assembled using one of the tubes provided 1 in the kit. 600 μL of the lysate/ethanol was applied onto the column and centrifuged for 1 minute at 14,000×g. The flow-through was discarded and the spin column was reassembled with its collection tube. 400 μL of the supplied wash solution was applied to the column and centrifuged for 1 minute at 14,000×g. The flow-through was discarded and the spin column was reassembled with its collection tube. The column was washed twice more by adding another 400 μL of wash solution and centrifuging for 1 minute at 14,000×g. The flow-through was discarded and the spin column was reassembled with its collection tube. The column was spun for two minutes at 14,000×g to thoroughly dry the resin and the collection tube was discarded. The column was assembled into a 1.7 mL elution tube provided with kit. 50 μL of elution buffer was added to the column and centrifuged for two minutes at 200×g followed by one minute at 14,000×g. The resulting purified RNA sample could be stored at −20° C. for a few days. For long 22 term storage of samples were stored at −70° C.

(1) miRNA Microarray and Data Analysis

Labelling

Purified RNA samples were labelled using a labelling kit from Agilent. The total RNA sample was diluted to 50 ng/μL in 1×TE pH 7.5. 2μï. of the diluted total RNA was added to a 1.5 mL micro-centrifuge tube and put on ice. Immediately prior to use, 0.4 μL 10×calf intestinal phosphatase buffer, 1.1 μL nuclease free water and 0.5 μL calf intestinal phosphatase were gently mixed to prepare a calf intestinal alkaline phosphatase master mix. 2 μL of the calf intestinal alkaline phosphatase master mix was added to each sample tube for a total reaction volume 4 μL, and was gently mixed by pipetting. The reaction volume was incubated at 37° C. in a circulating water bath for 30 minutes. 2.8 μL of 100% DMSO was added to each sample. Samples were incubated at 100° C. in a circulating water bath for 5-10 minutes and then immediately transferred to an ice bath.

10×T4 RNA ligase buffer was warmed to 37° C. and spun until all precipitate had dissolved. Immediately prior to use, 1 μL of 10×T4 RNA ligase buffer, 3 cyanine3-pCp and 0.5 μL T4 RNA ligase were gently mixed to make a ligation master mix and put on ice. 4.5 μL of the ligation master mix was added to each sample tube for a total reaction volume of 11.3 μL. Samples were gently mixed by pipetting and spun down. The samples were then incubated at 16° C. in a circulating waterbath for two hours. The samples were then dried using a vacuum concentrator at 45-55° C. and the samples were determined to be dry if, when the tube was flicked the pellets did not move or spread.

Hybridization

125 μL of nuclease free water was added to the vial containing lyophilised 10×GE blocking agent supplied with the Agilent Kit and mixed. The dried sample was resuspended in 18 μL of nuclease free water. 4.5 μL of the 10×GE blocking agent was added to each sample. 22.5 μL of 2×Hi-RPM Hybridization buffer was added to each sample and mixed well. The resulting samples were incubated at 100° C. for 5 minutes, and then immediately transferred to an ice waterbath for a further 5 minutes. A clean gasket slide was loaded into the Agilent SureHyb chamber base ensuring the gasket slide was flush with the chamber base. The hybridization sample was dispensed onto the gasket well ensuring no bubbles were present.

An array was placed active side down onto the SureHyb gasket slide and assembled with the SureHyb chamber cover to form an assembled chamber. The assembled chamber was placed into 1 a hybridization oven set at 55° C. and rotated at 20 rpm for 20 hours at that temperature.

The arrays were subsequently washed using the supplied GE wash buffers before being scanned.

(2) Quantitative Real-Time PCR

Quantitative real-time PCR is carried out in three stages. The first two stages, to synthesise cDNA from the total RNA samples, use the qScript miRNA cDNA synthesis kit (Quanta Biosciences). The third step, QPCR reactions, use the SYBR Green PerfeCTa Low Rox Reaction Mix (Quanta Biosciences).

Poly(A) Tailing Reaction

Total RNA samples (of between 100 ng and 1 μg) are aliquoted into fresh 0.5 ml tubes and made up to 7 μl with nuclease-free water. 2 μl of 5×PAP (Poly(A) Polymerase) Tailing Buffer and 1 μl of Poly(A) Polymerase is added to each tube, then the tubes vortexed and centrifuged. The samples are then incubated in a thermal cycler under the following conditions: 37° C. for 20 minutes, then 70° C. for 5 minutes. Following this reaction, samples are placed on ice.

cDNA Synthesis Reaction

A mastermix of RT is prepared so that each sample will receive 9 μl of miRNA cDNA Reaction Mix and 1 μl of qScript Reverse Transcriptase. 10 μl of this mix is added to each sample, then the tubes vortexed and centrifuged. The samples are then incubated in a thermal cycler under the following conditions: 42° C. for 20 minutes, then 85° C. for 5 minutes. Following this reaction, samples are placed on ice and then diluted 5-fold in 1×TE buffer. QPCR Reaction

A mastermix of SYBR Green reaction mix and primers is prepared so that each sample well will receive the following kit components:
10 ul of 2×SYBR Green PerfeCTa Low Rox Reaction Mix
0.4 μl of UA3PA Universal Reverse primer (10□M)
0.4 μl of miRNA-specific primer (10□M)
4.2 μl of nuclease-free water

To each well, 5 μl of cDNA is added. When all the wells are filled, the plate is sealed with plastic optical lids and centrifuged to remove air bubbles. The plate is loaded into the Agilent MX3005P thermocycler and processed under the following cycling conditions:
95° C. for 2 minutes
(95° C. for 5 seconds, 60° C. for 30 seconds)×40 cycles
Fluorescence data is collected at the end of every annealing/extension step

Data Analysis

Data from both of these techniques was normalised against the spike-in miRNA spots for each plate, allowing data from separate arrays to be compared. Normalised data was analysed using Principal Component Analysis, a standard technique well understood by those skilled in the art to identify correlations between miRNA expression profiles, and any grouping of data observed determined to be a 15 consequence of the action of the particular test condition in relation to the original cells on the expression of the individual miRNA.

FIG. 1 is a flow diagram of a method for obtaining an expression profile for micro RNA.

FIGS. 2A-2E the alterations in has-miR-210, hsa-miR1274a and hsa-miR-302c* between passage numbers identified by microarray analysis and confirmed by QPCR measurements of the mature microRNAs.

FIGS. 3A-3E show alterations in microRNA profiles in a serially-passaged human, tumour-derived cell lines (HeLa and MCF-7).

As can be seen in FIGS. 2A-2E, the results are clearly grouped and that this grouping is according to the passage number of the cells in which the miRNAs were expressed. In other words, it is possible to determine that the replicate samples of identically-passaged cells have similar but distinct miRNA expression profiles.

A database of miRNA expression patterns can be built up by carrying out many comparisons of cell passage number and analysing the resulting changes in miRNA expression. Such a database would enable identification of phenotypic drift in pluripotent stem cells, or cell lines used in bioprocessing and indicate a loss of optimal functionality, in the former case pluripotent potential, in the latter case productions of a desired macromolecule. Furthermore, building up a database of miRNA expression data may reveal a subset of certain miRNAs that are indicative of an unfavourable or undefined alterations to cell physiology. Once subsets of indicative miRNAs are identified, future testing of new cell lines can be carried out by looking at the expression profiles of the subset of indicative miRNA expression profiles and not the entire range of miRNAs produced by the cells. miRNAs may be ranked in order of the relevance of their expression levels for discriminating between biological interventions, or between groups of interventions known or hypothesized to have similar effects on cell physiology. miRNAs may be allocated a numerical value indicative of the relevance of their expression levels for discriminating between interventions, or between groups of interventions known or hypothesized to have similar effects on the cells. For example, the numerical value may be related to the contribution of the expression level of a miRNA to the variance of principle components. As an alternative to, or in addition to, the comparison of miRNA expression profiles using statistical methods such as principal component analysis, the effect cell culture passages on the expression of each of a limited group of miRNAs (for example, 10-50) may be identified and used to assign a code, selected from a group of codes, to the effect of the biological intervention on the expression of each respective miRNA. The resulting codes may be compared to identify similarities in effect.

For example, for comparison (e.g. cell passage number) a 3-digit binary number may be allocated as a code to each ranked miRNA based on:
1. If expression of the miRNA is unchanged (within normal limits of experimental variability) in response to the biological intervention, the first bit is set to 0. If expression has changed significantly, the first bit is set to 1.
2. If a change in expression level was identified and the change was an increase, the second bit is set to 1. If the change resulting from the biological intervention was a decrease, the second bit is set to 0.
3. If the change in expression level was more than 4-fold, the third bit is set to 1, otherwise it is set to 0.

Thus, the effect of a difference between cell passages or culture conditions on the expression of a miRNA is allocated a code having one of five possible values:
4. No change 2 in expression—000
5. Large increase in expression—111
6. Small increase in expression—110
7. Large decrease in expression—111
8. Small decrease in expression—100

The effect extended time in culture (i.e., an increase in passage number) on the expression level of a group of miRNAs may be characterised by the associated code, permitting identification of changes in expression level not immediately apparent from principal component analysis, permitting alternative methods of scoring the similarity of test conditions or interventions and rendering the resulting expression data comprehensible by visual inspection.

Another way to characterise the effect of a cell maintenance regime and to determine correlations between the effects on miRNA expression of different biological interventions is to carry out an expression assay to determine the effects of an intervention on the expression of each of a group (of typically 10 to 50) miRNAs and to rank the miRNAs in that group in order of the effect, for example, in order from the miRNA in the group which has the largest increase in expression to the miRNA in the group which has the largest decrease in expression, or vice versa. The resulting rankings are indicative of the effects of particular test point or interventions. Thus, the effect of other interventions on the group of miRNAs may be measured and the miRNAs in the group ranked in order of the effect. The resulting rankings may be compared to enable correlations between the effects of interventions to be identified.

A kit comprising plates operable to test the subset of indicative miRNAs may be provided to significantly increase the efficiency and speed with which the effect of cell passage and/or interventions can be screened for potential novel therapeutic applications.

Further variations and modifications may be made within the scope of the invention herein disclosed.

References
9. Xie, X., et al., Systematic discovery of regulatory motifs in human promoters and 3′-UTRs by comparison of several mammals. Nature, 2005. 434(7031): p. 338-45
2. Lim, L. P., et al., Microarray analysis shows that some microRNAs downregulate large numbers of target mRNAs. Nature, 2005. 433(7072): p. 769-73
3. Calin, G. A., et al., MicroRNA profiling reveals distinct signatures in B cell chronic lymphocytic leukemias. Proc Natl Acad Aci USA, 2004. 101 (32): p. 11755-60 EXAMPLE 2

Summary
10. MicroRNA profiling of serially-passaged stem cells reveals differences in cells assessed to be ‘identical’ populations using flow cytometry and a commercial kit assessing cell surface and internal protein antigen markers of pluripotency and differentiation, as shown in FIGS. 4A-4D.
11. micro-RNAs can be used to monitor the directed differentiation of hESC to erythrocytes by comparing miRNA profiles from two populations of CD34+ cells derived by directed differentiation of human embryonic cell lines (hESCs) for comparison with the equivalent developmental stage of adult CD34+ haematopoietic stems cells (HSCs;), as shown in FIGS. 5A and 5B.

Methods.

These are outlined in Example 1 above (see section headed “Flow Cytometry” and “Data analysis”—in particular, PCA).

The hierarchical clustering and heatmap visualisation of the data were achieved using Qlucore Omics Explorer (Qlucore AB).

A volcano plot is a graphical representation of that is used to quickly identify changes in large datasets composed of replicate data. It plots significance versus fold-change on the y- and x-axes, respectively. The volcano plot was generated using the results of an ANOVA analysis for the hESC datasets. Both the ANOVA analysis and Volcanoes plot were generated using Partek's Genomic Suite (Partek, Inc).

Results and Discussion.
12. Identification of miRNA differences in pluripotent hESC cell populations otherwise assessed to be identical.

Roslin Cellabs utilised a human embryonic stem cell line, RCM1. Cells were obtained at mid-passage 51 (P51) and a late passage (P103), where an individual passage (i.e. the period between cell sub-culturing) is about 1-week. The cells were grown for up to three passages post-resuscitation from liquid nitrogen storage in order to generate sufficient cells for analysis by flow cytometry and miRNA profiling.

Flow Cytometry, MicroRNA Profiling and Data Analysis

The cells from each passage were analysed using Flow Cytometry carried out by Roslin Cells. This analysis, as shown in FIG. 4A, suggests that both cell populations are indistinguishable for the pluripotentcy and differentiation markers used in the commercial test. However, as can be seen in FIG. 4B below, the biological replicates (n=3) at each passage clearly group together according to the passage number of the cells in which the miRNAs were expressed. In other words, it is possible to determine that the replicate samples of identically-passaged cells have similar but distinct miRNA expression profiles.

There were 5 differentially-expressed miRNAs with a fold-change difference of 2 or more, as shown in FIG. 4C, and the identity of these miRNAs are given in FIG. 4D.

2. Monitoring hESC-Derived and Adult Haematopoietic Stem Cells Directed to Differentiate to Erythrocytes.

A PCA of the top 50 most variable miRNA transcripts is shown in FIG. 5A below. The samples cluster distinctly based on cell type and stage, which is also evident from the heatmap in FIG. 5B. For stage 1, the hESC and Adult HSC categories occupy separate spaces on the PCA plot, implying that these cell types have distinctly different properties. At stage 2, however, the hESC and Adult HSC categories are largely grouping together, demonstrating that the miRNA profiles of the samples are highly similar.

The following sample groups were analysed:
hESC stage 0: Undifferentiated hESC
hESC stage 1: hESC at day 10 of the differentiation protocol
hESC stage 2: hESC at day 24 of the differentiation protocol
Adult HSC stage 1: Adult HSC cells at a differentiation stage equivalent to that of hESC stage 1
Adult HSC stage 2: Adult HSC cells at a differentiation stage equivalent to that of hESC stage 2 (14 days after induction of differentiation) Embodiments of this invention may relate to:

A method comprising steps of:
i. Growing cell lines as serially passaged cultures and at each passage where the cells are sampled, determining the microRNA expression profile, for example by microarraying, following a defined intervention or where there is no change to the growth conditions
ii. Define using a appropriate statistical test, for example Principal Components Analysis, separation between samples based on passage number, alterations to growth conditions, treatment with drugs or other external factors, transfection/viral transduction of gene(s)
iii. Determining the microRNAs which define the variation between the test conditions These miRNAs can inform the ‘drift’ of the cells from optimally pluripotent, optimally differentiating and/or optimally growing cells population and/or those safe for their purpose in bioprocessing, drug discovery or regenerative medicine i.e. reveal key information on the identity, purity, potency or safety (tumourigenicity of stem cells, microbiological contamination) of the cell population

Where the cells are mammalian (possibly human and/or rodent) undifferentiated, pluripotent, embryonic stem cells or iPS cells (where iPS cells (induced pluripotent stem cells) are defined as adult somatic cells which have been reprogrammed by direct expression of exogenous cDNAs/mRNAs/miRs from one or more transduced vectors). In combinations that may include chemical entities necessary for their production.

Where the cells are a mixture of one or more of the primary germ layers or progenitor cells derived from hESC or iPS cells

Where the cells are mirPS cells (from Mello Inc) or other cells reprogrammed by direct expression of exogenous miRNA(s) form one or more transduced vectors.

Where the biological system represents plasmid-based assay systems, controllably inserted into the hESC genome and have them actively express in pluripotent as well as in differentiated lineages derived from the genetically engineered cells.

Where tissue-specific stem cells are used to produce one or more terminally differentiated lineages following exposure to biological factors and/or chemical entities that direct differentiation

Where the cells are human or animal multipotent mesenchymal stem cells or any other adult stem cell population

Where the cells are primary cell cultures derived from human or animal tissues

Where the cells are established cell lines with and without genetic modifications (e.g. with virus or plasmid-based expression of an exogenous enzyme, protein or peptide)

Where the change in growth conditions includes alterations in cell matrix, including switching from 2-dimensional to 3-dimensional culture systems, cell media composition, addition of xenogenic components, drugs, excipients and chemicals, including those used for cosmetics, exposure to biological agents & their biosimilars, variations physical conditions (e.g temperature, radiation etc.).

Monitor commitment towards specific lineages following exposure to small molecules and biological factors (biologies or biosimilars), either alone or in combination.

For bioprocessing application specifically, monitor the effects in alterations dues to pH, osmolality etc.

Others relating to the way the microRNAs are changing—positive or negative correlations as well as combinations of microRNA changes i.e. the pattern of miR changes defines the alteration to cell phenotype.

1.-15. (canceled)

16. A method of quality assessing stem cells cultured from an in vitro cell culture system for use in stem cell therapy, said method comprising the steps of:
providing a stem cell sample from the stem cell culture,
providing an assay and/or reagents for obtaining microRNA profiles from the stem cell samples and comprising a predetermined panel of microRNAs,
providing a reference expression profile of the predetermined panel of microRNAs which is derived from a stem cell sample that conforms to a predetermined standard for the use of stem cells in stem cell therapy, the predetermined panel of microRNAs selected as being proxy to certain quality characteristics consistent with the predetermined standard for the use of stem cells in stem cell therapy,
employing a microRNA expression assay with the stem cell sample for the predetermined panel of microRNAs to obtain a microRNA expression profile for the stem cell sample,
comparing the microRNA profile of said stem cell sample with the reference microRNA expression profile,
determining from the comparison a quality assessment of the stem cell sample for use in stem cell therapy and, dependent upon the quality assessment, determining the suitability of the stem cell sample for use in stem cell therapy, and
supplying stem cells from the stem cell culture for use in stem cell therapy.

17. The method according to claim 16 wherein the panel includes microRNAs that are markers of undesirable or uncharacterized alterations in the cell system.

18. The method according to claim 16, wherein the qualities being assessed are the maintenance of cell identity and functional capability.

19. The method according to claim 16, wherein the predetermined panel of microRNAs includes microRNAs derived from cells of known identity and/or known functional capability and determined to be a marker of the cells identity and/or functional capability.

20. The method according to claim 16, wherein the qualities being assessed are selected from one or more of phenotype, low contamination level and viability in accordance with a predetermined standard.

21. The method according to claim 20, wherein the predetermined panel of microRNAs includes microRNAs derived from one or more cells of known phenotypes, contamination levels selected from no or low contamination levels and moderate or high contamination levels, and viability, and determined to be a marker of a desired and an undesired quality of one or more of phenotype, contamination level and viability.

22. The method according to claim 16, wherein the quality being assessed is potency of stem cells.

23. The method according to claim 22, wherein the predetermined panel of microRNAs includes microRNAs derived from stem cells of known potency and determined to be a marker between a desired and an undesired stem cell potency.

24. The method according to claim 16, wherein the reference expression profile of the panel of microRNAs which is derived from a stem cell sample that conforms to a predetermined standard for the use of stem cells in stem cell therapy is a reference expression profile of a stem cell population cultured according to a particular protocol which stem cell population conforms to a predetermined standard for use in stem cell therapy.

25. The method according to claim 24, wherein the predetermined panel of microRNAs include at least one microRNA as a marker for altered culture conditions over the particular culture protocol.

26. The method according to claim 25, wherein the step of providing the reference microRNA expression profile comprises providing a reference stem cell sample that conforms to a predetermined standard for the use of stem cells in stem cell therapy and employing a microRNA expression assay with the reference cell sample for the panel of microRNAs contemporaneously with the stem cell sample being quality assessed.

27. The method according to claim 16, wherein the step of providing the reference microRNA expression profile comprises providing a database of the microRNA expression profiles derived from a stem cell sample that conforms to a predetermined standard for the use of stem cells in stem cell therapy.

28. The method according to claim 16, wherein the step of comparing the microRNA expression profile of the cell sample with the reference expression profile comprises identifying correlations between microRNA profiles.

29. The method according to claim 28, wherein the correlations may comprise positive and/or negative correlations between the expression of one or more microRNAs.

30. The method according to claim 29, wherein a positive correlation is an expression of a microRNA in the sample to be quality assessed and in the reference microRNA profile and a negative correlation is expression of a microRNA in the sample to be quality assessed which exhibits differential expression in the reference microRNA profile.

31. The method according to claim 16, wherein the stem cells are induced pluripotent stem cells.

32. The method according to claim 16, wherein the stem cells are mesenchymal stem cells.

33. A method of quality assessing stem cells cultured from an in vitro cell culture system for use in stem cell therapy, said method comprising:
employing a microRNA expression assay with a stem cell sample from a stem cell culture for a predetermined panel of microRNAs, selected as being proxy to certain quality characteristics consistent with the predetermined standard for the use of stem cells in stem cell therapy, to obtain a microRNA expression profile for the stem cell sample,
wherein a comparison of the microRNA profile of said stem cell sample with a reference microRNA expression profile of the predetermined panel of microRNAs which reference microRNA expression profile is derived from a stem cell sample that conforms to a predetermined standard for the use of stem cells in stem cell therapy, allows for a determination of a quality assessment of the stem cell sample for use in stem cell therapy, and depending upon the quality assessment, allows for a determination of the suitability of the stem cell sample for use in stem cell therapy, and
optionally supplying stem cells from the stem cell culture for use in stem cell therapy.

34. A kit comprising a database of non-coding RNA profiles obtained from cells having known characteristic(s) and/or a known profile and an assay and/or reagents for obtaining non-coding RNA profiles from cells to be characterized, profiled and/or quality controlled.
